(19) United States
(12) Patent Application Publication
(10) Pub. No.: US 2025/0041593 A1
(43) Pub. Date: Feb. 6, 2025

(54) ASSEMBLY FOR THE TRANSVASCULAR STIMULATION OF PART OF THE AUTONOMIC NERVOUS SYSTEM OF A HUMAN BODY, IN PARTICULAR FOR TRANSVASCULAR RENAL NERVE STIMULATION OR TRANSVASCULAR STIMULATION OF THE CAROTID BODY

(71) Applicant: ELECTRODUCER, Grenoble (FR)

(72) Inventors: Benjamin FAURIE, MEYLAN (FR); Marie-Claude MORICE, Paris (FR)

(21) Appl. No.: 18/717,653

(22) PCT Filed: Dec. 9, 2022
(86) PCT No.: PCT/EP2022/085221

§ 371 Date: Jun. 7, 2024

(30) Foreign Application Priority Data
Dec. 10, 2021 (FR) FR2113336

(51) Int. Cl. A61N 1/05; A61M 25/00; A61M 25/06; A61N 1/36; A61N 1/372

(52) U.S. Cl. CPC A61N 1/0551; A61N 1/36007; A61N 1/3605; A61N 1/372; A61M 25/0082; A61M 2025/0681; A61M 2210/12

(57) ABSTRACT

The application relates to an assembly for the transvascular stimulation of part of the autonomic nervous system of a human body, in particular for transvascular renal nerve stimulation or transvascular stimulation of the carotid body. The application essentially consists of an assembly for unipolar transvascular stimulation from inside an artery, which enables nerve stimulation of the region(s) of the autonomic nervous system around the artery, it being possible for this stimulation to be either rapid or for mapping purposes.

### TECHNICAL FIELD

The present invention relates to a kit for transvascular stimulation of part of an autonomic nervous system of a human body.

Here, and in the context of the invention, “autonomic nervous system” means the nervous system which directs the internal organic functions and does not manage the voluntary mechanisms of the human body and thus differs from the somatic system which concerns the relations of the body with the outside world. The autonomic nervous system essentially innervates the internal organs, and the sensory neurons of this system carry information concerning visceral functions to the central nervous system. Three categories are usually distinguished in the autonomic nervous system: the sympathetic nervous system, the parasympathetic nervous system, and the enteric nervous system.

Here, and in the context of the invention, “blood vessel” means a vein or an artery.

The invention aims in the first instance to propose a simple and effective solution for transvascular stimulation, for particularly rapid diagnosis and/or for mapping of a nerve region, in particular with a view to a surgical or percutaneous intervention.

Although described with reference to a use for renal nerve stimulation, the invention applies to any other use for stimulation of part of the autonomic nervous system of the human body. In particular, the invention also applies to nerve stimulation of the carotid body.

### PRIOR ART

Renal sympathetic denervation by catheter appears promising for the treatment of diseases/deficiencies that primarily include hypertension, but also heart failure, cardiac arrhythmias, diabetes, and obstructive sleep apnea.

In particular, hypertension is a major health problem worldwide, associated with a significantly increased risk of strokes.

Although most patients agree to long-term treatment with drugs effective in treating their hypertension, a large proportion of them remain unobserved as regards the possible side effects and/or the inability of the drug to achieve blood pressure targets despite maximum tolerated regimens. In some cases, high blood pressure is resistant to drug therapy.

Thus, several new treatment strategies based on the use of medical devices have recently been developed to help control blood pressure by modulating the sympathetic nervous system.

Of these, renal sympathetic denervation has been the one most studied. Renal denervation is a technique involving superficial stimulation of the afferent and efferent nerves situated around the renal arteries, either using a single-electrode catheter connected to a radiofrequency generator or using a multi-electrode catheter connected to an ultrasound generator, or using a catheter provided at its distal end with needles for injecting an alcohol-based liquid product.

One of the major challenges of this denervation technique lies in the lack of markers along the renal arteries identifying the exact location of the part of the nerves to be destroyed and thus in determining the regions to be treated in order to optimize the effectiveness of the denervation treatment.

The first trials of renal artery denervation by catheter for resistant hypertension and using a radiofrequency generator did not show consistent clinical efficacy, widely considered to be primarily due to procedural factors that limited the assessment of the completeness of the denervation obtained.

According to the authors of [1], the difficulty of predictability of renal denervation and the controversy surrounding the paradigm of renal denervation as an antihypertensive treatment owe much to the absence of means of evaluating the denervation during the procedure, that is to say a measurable criterion for confirming the success of the procedure.

Some authors have shown that:
the aorticorenal ganglia can be localized by a transvascular approach with high-frequency unipolar electrical stimulation at 10 Hz, under a current of 25 mA, of the inferior vena cava or aorta, or the branches thereof,
stimulation of the aorticorenal ganglia induces arterial vasoconstriction and a concomitant increase in blood pressure,
renal denervation blocks the activation of the afferent renal nerve and subsequently abolishes the renovascular responses to the stimulation of the aorticorenal ganglia.

The publication [2], which mentions an electrical stimulation at 20 Hz with pulses of 5 ms under a current of 10 mA, makes a similar observation.

For their part, the authors of the publication [3] use a multi-electrode catheter marketed under the name “ConfidenHT™” with a distal end that deploys in the form of an ellipsoid with four branches, each supporting an electrode, when a button on a handle is pressed. This catheter is very invasive because its diameter is considerable, of the order of 2.67 mm (8 French), and, although the authors mention the possibility of nerve mapping using such a catheter, this remains to be demonstrated, in view of the deployed shape that cannot be truly moved within the artery in order to at least easily reach other nerve sites. The placement of this catheter is also not easy to carry out.

Thus, the devices proposed to date can be considered too invasive and are not necessarily reliable for ensuring a mapping of a renal artery and/or are not simple to use.

There is therefore a need to improve renal nerve stimulation devices, in order to overcome the above drawbacks.

More generally, there is a need for a medical device which permits nerve stimulation of part of the autonomic nervous system and which is simple, effective in particular for the purposes of mapping or monitoring the effectiveness of a denervation, and non-invasive.

The aim of the invention is to respond at least in part to this need.

### DESCRIPTION OF THE INVENTION

To do this, the invention relates, according to a first alternative, to a kit for transvascular stimulation of part of an autonomic nervous system of a human body, comprising:
an introducer or a guide catheter, intended to be introduced into a blood vessel of the human body;
a sleeve adapted to be fitted around the introducer or guide catheter, the sleeve being made of electrically conductive material over at least part of its outer periphery so that, when the guide catheter is introduced into the blood vessel, the conductive periphery of the sleeve is in contact with the subcutaneous tissue of the human body or with the wall of the blood vessel, the sleeve additionally being connected to an electrical connection which is itself connected to an electrode of an electrical pulse generator external to the body;
an elongate electrical stimulation element intended to be inserted into the introducer or into the introducer sheath of the guide catheter, the elongate element comprising:
   an electrically conductive distal portion, atraumatic to the blood vessel, and configured to be shaped into a shape with at least one point of contact with the blood vessel when it is in its stimulating position facing the part of the autonomic nervous system around the blood vessel,
   an electrically insulating central portion, and
   an electrically conductive proximal portion for connection to the other electrode of the electrical pulse generator, so as to effect the electrical stimulation through the shaped distal portion.

According to an advantageous configuration, the electrode of the electrical pulse generator to be connected to the electrically conductive sleeve fitted around the introducer or the introducer sheath of the guide catheter is the anode, while the one to be connected to the proximal portion of the elongate element is the cathode.

According to an advantageous variant, the electrically conductive sleeve is composed of a one-piece component made of conductive material, such as carbon.

According to this variant, the sleeve is preferably composed of a sheath having an electrically conductive coating, such as a carbon coating, on its outer periphery.

According to this variant, the sleeve is preferably elastic so as to be able to fit on peripheral arterial or venous catheter sheaths of different diameters, typically of external diameters of between 1.6 and 20 mm.

According to a second alternative, the invention also relates to a kit for transvascular stimulation of a part of an autonomic nervous system of a human body, comprising:
an introducer or a guide catheter intended to be introduced into a blood vessel of the human body, the introducer or the guide catheter comprising at least one tubular introducer sheath and at least one electrically conductive element, of which a distal portion is exposed over at least part of the outer periphery of the sheath so as to be in contact with subcutaneous tissue of the body or with the wall of the blood vessel, and of which a proximal portion, accessible from outside of the body (C), comprises an electrical connection so as to serve as a connection to an electrode of an electrical pulse generator to the body;
an elongate electrical stimulation element intended to be inserted into the introducer or into the introducer sheath of the guide catheter, the elongate element comprising:
   an electrically conductive distal portion, atraumatic to the blood vessel, and configured to be shaped into a shape with at least one point of contact with the blood vessel when it is in its stimulating position facing the part of the autonomic nervous system around the blood vessel,
   an electrically insulating central portion, and
   an electrically conductive proximal portion for connection to the other electrode of the electrical pulse generator, so as to effect the electrical stimulation through the shaped distal portion.

According to an advantageous configuration, the electrode of the electrical pulse generator to be connected to the introducer or to the guide catheter is the anode, while the one to be connected to the proximal portion of the elongate element is the cathode.

According to an advantageous variant, the electrically conductive element of the introducer or of the guide catheter is a wire or a metal strip which is housed at least partly within the thickness of the sheath and of which a distal portion is exposed at the outer periphery of the sheath.

Preferably, the cross section of the wire or of the metal strip is between 0.25 and 5 mm².

According to a first advantageous embodiment, the elongate element is a rectilinear wire, the distal portion of which comprises the contact shape, which extends radially along the axis of the wire.

Several advantageous alternatives for producing the contact shape are possible.

Thus, the contact shape can include:
at least one loop at the end of the wire or in proximity to the straight end of the wire;
at least one ring, preferably at least two adjacent rings, in proximity to the straight end of the wire;
at least one free strand, preferably four strands at 90° from each other, in proximity to the straight end of the wire.

According to a second advantageous embodiment, the elongate element comprises a guidewire, and a tube made of shape-memory alloy arranged around the guidewire and temporarily attached to the guidewire so that, once its distal portion has reached its stimulation position, withdrawal of the guidewire toward the outside of the body causes the distal portion of the tube to adopt the contact shape.

Preferably, the contact shape of the distal portion of the tube is a helix shape.

According to an advantageous variant embodiment, the distal portion of the elongate element comprises one or more radiopaque markers.

The kit can be a kit for transvascular renal nerve stimulation, and furthermore a renal denervation kit.

It can also be a kit for stimulation of the carotid body, and furthermore a kit for delivery of a stent or a balloon for carotid dilation.

The invention also relates to a method for nerve stimulation of part of a patient's autonomic nervous system and, if necessary, for surgical intervention, comprising the following steps:
i/ fitting a sleeve around a guide or introducer catheter, the sleeve being made of electrically conductive material over at least part of its outer periphery and further comprising an electrical connection to an electrode of an electrical pulse generator external to the body;
ii/ introducing a guide catheter or the introducer into a femoral or radial artery of a human body, so that the conductive periphery of the sleeve is in contact with the subcutaneous tissue of the body or with the wall of the artery or vein;
iii/ introducing a wire into the introducer sheath of the guide catheter or of the introducer, the wire comprising:
   an electrically conductive distal portion, atraumatic to the blood vessel, and configured to be shaped into a shape with at least one point of contact with the blood vessel when it is in its stimulating position facing the part of the autonomic nervous system around the blood vessel,
   an electrically insulating central portion, and
   an electrically conductive proximal portion serving for connection to the other electrode of the electrical pulse generator, so as to effect the electrical stimulation through the shaped distal portion;
iv/ direct unipolar nerve stimulation on the wire by means of the external electrical pulse generator;
v/ if necessary, surgical intervention in one or more regions stimulated according to step iv/.

Thus, the invention consists of a kit for unipolar transvascular stimulation from within an artery, which permits nerve stimulation of the region or regions of the autonomic nervous system around the artery, which stimulation can be either rapid or for mapping purposes.

This nerve stimulation can notably be a transvascular (artery or vein) renal stimulation of the regions of the sympathetic system that are likely to generate arterial hypertension. It is thus possible to perfectly identify the nerve region(s) to denervate in order to reduce or eliminate high blood pressure. This stimulation also makes it possible to verify the effectiveness of the denervation performed previously.

It can also involve transvascular (artery or vein) stimulation of the carotid body. By performing rapid stimulation, it is possible to observe induced temporary hypotension and/or bradycardia (cardiac slowdown). This makes it possible to predict bradycardia or arterial hypotension prior to a carotid angioplasty procedure and therefore to be able to anticipate it and administer drugs in order to avoid this danger.

The guide catheter or introducer of the stimulation kit according to the invention may be entirely conventional for the anatomy of the blood vessel to be diagnosed.

The electrical stimulation element, notably a simple wire conductive in its proximal and distal portions, has an atraumatic contact shape that makes contact with the specific region(s) of the blood vessel (artery or vein) facing the endings of the nervous system to be stimulated, which is around the vessel.

In addition, the required intensity of nerve stimulation is low. Typically, the intensity of the alternating current delivered can range from 10 to 25 mA for approximately 10 to 60 seconds, at a frequency ranging from 200 to 800/min (10 to 20 Hz).

The handling of all the components of the kit is simple: a surgeon in charge of the operation can thus easily connect the electrode, typically the anode, of the electrical pulse generator to the conductive sleeve fitted around the catheter guide and then, as usual, connect the other electrode, typically the cathode, to the electrical stimulation wire.

In summary, the advantages of a transvascular nerve stimulation kit for diagnostic purposes are numerous, among which may be mentioned:
a medical device that is simple to put in place;
a reliable transvascular nerve stimulation device which makes it possible to very precisely map the nerve regions(s) of a patient's autonomic nervous system;
in addition to the mapping before a denervation or carotid treatment procedure, a device that makes it possible to verify the effectiveness of the intervention during and after same,
possibility of implementation on any existing guide catheter, since all that is needed is for a conductive sleeve to be fitted around this catheter, before its introduction into a vein or artery to be stimulated, and for an electrical connection to be made to the wire inserted in the catheter;
possibility of using a thinner, less invasive catheter, typically with a diameter of 6 French and not 8 French as in the devices according to the prior art, and especially upstream of a possible surgical procedure;
possibility of being able to use the wires or guides familiar to cardiologists or surgeons.

The only relative constraint of the sleeve is at the start of the intervention during preparation and consists in fitting the sleeve around the introducer sheath of the guide catheter or the introducer.

However, this procedure is very simple and easy to implement and can be carried out by an assistant or nurse, who does not need to have any special skills for this task.

Further advantages and features will become clearer on reading the detailed description, provided by way of non-limiting illustration, with reference to the following figures.

### BRIEF DESCRIPTION OF THE DRAWINGS

FIG. 1 is a schematic view illustrating the use of a renal nerve stimulation kit according to the invention with a guide catheter and a stimulation sleeve according to the invention, the guide catheter being introduced directly into a peripheral artery of a patient.

FIG. 2 shows the arrangement of the electrical stimulation wire of the kit according to FIG. 1 within a renal artery for nerve stimulation of the surrounding nervous system.

FIG. 3 is a perspective view of an electrically conductive sleeve according to the invention intended to be fitted around a guide catheter as shown in FIGS. 1 and 2.

FIG. 4 is a partial longitudinal sectional view showing a sleeve according to FIG. 3 fitted around a guide catheter.

FIGS. 5A to 5D are schematic views showing various alternative embodiments of the contact shape of the distal end of an electrical stimulation wire according to the invention.

FIG. 6 schematically shows another variant embodiment of a wire with an electrical stimulation tube according to the invention, before and after deployment of the tube.

FIG. 7 schematically shows different electrical stimulation positions for mapping the renal nervous system around a patient's renal artery.

FIG. 8 is a schematic view illustrating the use of a carotid nerve stimulation kit according to the invention with a guide catheter and a stimulation sleeve according to the invention, the guide catheter being introduced directly into a peripheral artery of a patient.

### DETAILED DESCRIPTION

In the following description and throughout the present application, the terms “distal” and “proximal” are used with reference to the body of a patient for whom renal nerve stimulation or carotid nerve stimulation is followed, if necessary, by an intervention. Thus, the distal end of a wire is the end situated furthest inside the patient during nerve stimulation for diagnosis.

It will be noted that the various elements are not necessarily shown to scale.

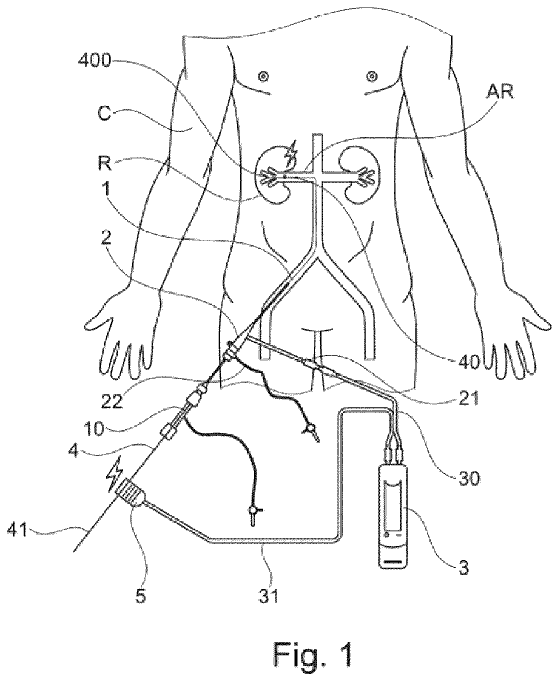

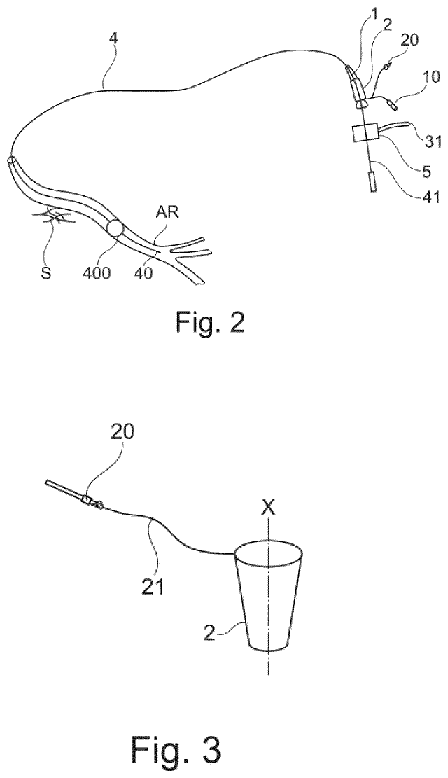

FIGS. 1 and 2 show a diagnostic kit according to the invention.

A guide catheter 1 is introduced into a femoral artery.

Such a guide catheter 1 can be of small diameter. It complies with the standards relating to existing peripheral intravascular catheters. The catheter 1 can comprise a flushing device 10 with stopcocks, commonly referred to as a “valve” or “Y-piece”, for flushing the inside of the catheter 1 with a suitable flushing liquid or for injecting contrast medium.

An electrically conductive sleeve 2 is fitted directly around the introducer or the guide catheter 1. The sleeve 2 is connected to an electrical connection 20, which is itself connected to an electrode, typically the anode of an electrical pulse generator 3, external to the body C, by way of an electrical supply wire 30.

An electrical stimulation wire 4 is introduced into the sheath 11 of the guide catheter 1. The length of this wire 4 is typically between 180 and 300 cm.

As is shown in FIG. 2, the wire 4 comprises an electrically conductive distal portion 40, atraumatic for the renal artery, and configured to be shaped according to a shape 400 with at least one point of contact with the renal artery, so as to stimulate the sympathetic nervous system S that surrounds it.

In the example illustrated in FIG. 2, the contact shape 400 can be a single loop, which can even be located at the end of the proximal portion.

Once deployed, this loop 400 has a substantially circular shape, centered on the axis of the wire, which allows circumferential contact with the blood vessel, here the renal artery, once the wire 4 is in its stimulation position. Typically, the diameter of this circular shape 400 varies from 3 to 8 mm in diameter.

The central portion of the wire 4, at the very least the part just close to the contact shape 400 and toward the proximal portion 41 of the wire 4, is electrically insulated, so as not to conduct the stimulation current against the vessel wall.

The proximal portion 41 of the wire is electrically conductive and serves as a connection to the other electrode, typically the cathode of the external electrical generator 3.

To ensure the electrical connection, a crocodile-type clip 5 is fixed by clipping onto the distal portion 41 of the wire 4. This clip 5 is connected to the anode of the electrical generator 3 by way of an electrical supply wire 31.

The diameter of the wire 4, including its contact shape 400, can be compatible with the internal lumen of a sheath 11 of a guide catheter of 1.4 mm diameter (5 French) or 1.8 mm (6 French).

A sleeve 2 according to the invention is shown in FIG. 3. It is in the form of a tube, at least part of the outer periphery of which is electrically conductive.

This conductive part is connected to an electrical connection 20, typically in the form of a connector, by way of an electrical supply wire 21. The sleeve 2, the connector 20 and the electrical supply wire 21 can advantageously constitute a kit ready to be fitted around a catheter 1 and connected to an electrode of the electrical generator 3.

The sleeve 2 can be cylindrical or frustoconical in shape. Its shape matches as closely as possible the outer shape of the introducer sheath of a guide catheter 1.

The sleeve 2 can comprise a flushing device 22 with stopcocks, commonly referred to as a “flush”, integrated to flush the inside of the catheter or of the introducer 1 by means of a suitable flushing liquid.

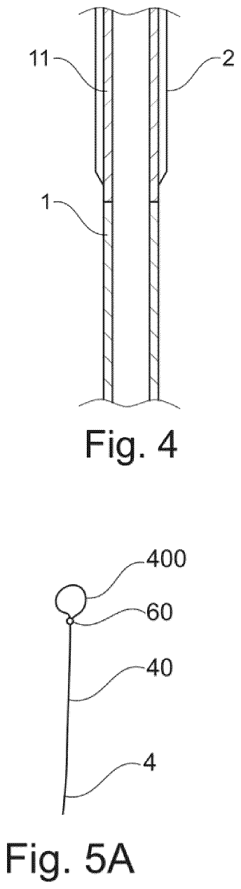

As can be seen in FIG. 4, the thickness of this sleeve 2, typically of the order of a millimeter or less, adds only a small extra thickness to the sheath 11 of the introducer or of the guide catheter 1 and therefore does not hinder the advance of the latter during its introduction into a peripheral artery V.

The sleeve 2 can be in the form of a fully conductive one-piece component or in the form of a component coated with an electrically conductive coating. Carbon may advantageously be chosen as an electrically conductive material.

According to an advantageous variant, the sleeve 2 can be designed so that it is elastic and can thus adapt to any existing size of peripheral arterial catheter.

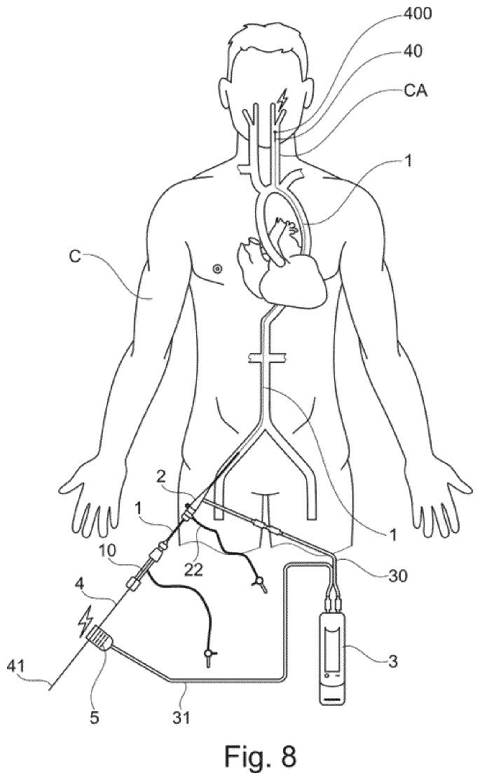

In practice, a surgeon or interventionist wishing to perform renal nerve stimulation (FIG. 1) or carotid nerve stimulation (FIG. 8), as explained below, begins by placing the conductive sleeve 2 around the introducer or a guide catheter 1. Since the placement of the conductive sleeve is very simple and easy, it can be done by an assistant or nurse without the need to adopt any particular technique.

The placement is achieved once the sleeve 2 is fitted and arranged around the introducer sheath of the guide catheter 1 in such a way that said sleeve 2 touches either a subcutaneous region of the patient or the wall of the peripheral artery or vein of the patient.

Once this placement has been carried out, the electrical connection 20 can be connected directly to the anode of an external cardiac stimulator 3 by way of a connecting wire 30.

Usually, a clip 5, such as a crocodile clip 5, can in turn be fixed by clipping on the proximal portion 41 of the wire 4 inserted into the sheath 11 of the peripheral catheter 1. This clip 5 is connected to the cathode of the external electrical generator 3 by way of a connection wire 31.

Thus, nerve stimulation of the sympathetic nervous system S through the renal artery AR can take place by unipolar electrical stimulation between the cathode electrically connected to the wire 4 and the anode electrically connected to the sleeve 2 according to the invention around the guide catheter 1.

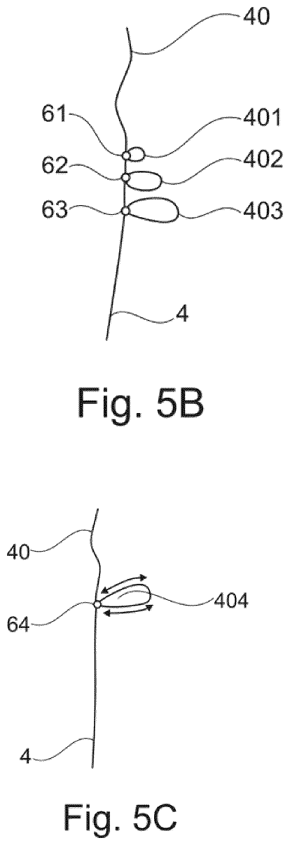

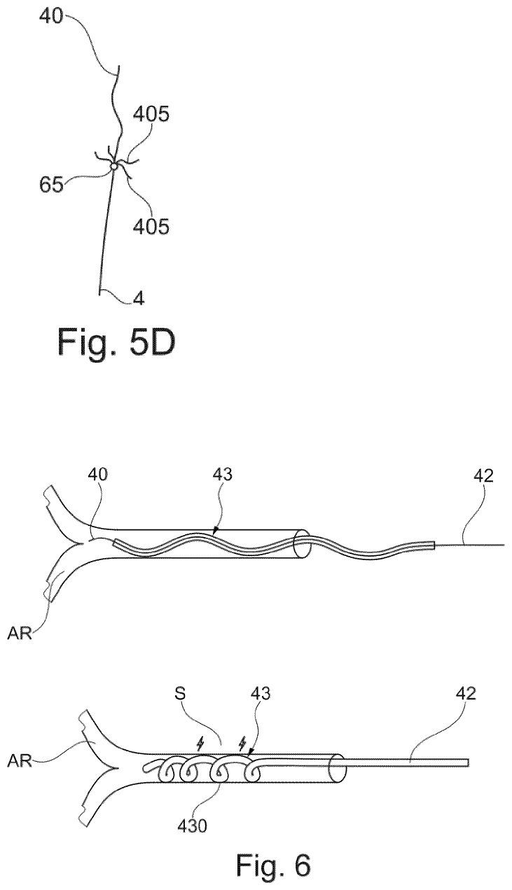

Various alternatives illustrated in FIGS. 5A to 5D can be provided for producing the contact shape in the proximal portion 40 of the wire 4.

FIG. 5A concerns a contact shape which is a loop 400 at the end of the distal portion 40 of the wire 4. One or more radiopaque markers (graduations) 60 are advantageously implanted at the base of the loop 400 of the wire 4 in order to facilitate angiographic identification.

FIG. 5B concerns a contact shape comprising three adjacent rings 401, 402, 403, close to the straight end of the wire 40. A radiopaque marker 61, 62, 63 is advantageously implanted at the base of each of these three rings 401, 402, 403. The radiopaque markers can be spaced every centimeter apart, for example.

FIG. 5C concerns a contact shape with a single ring 404 close to the straight end of the wire 40. One or more radiopaque markers 64 are advantageously implanted at the base of the single ring 404.

FIG. 5C concerns a contact shape with four strands 405 at 90° from each other, close to the straight end of the wire 40. A radiopaque marker 65 is advantageously implanted at the junction point of these strands 405.

Each of these variants can be used by the practitioner, for example depending on the anatomy of the patient.

An alternative embodiment of the electrical stimulation element in place of a single wire 4 is shown in FIG. 6.

In this alternative, the electrical stimulation element 4 comprises a guidewire 42 and a tube 43 made of shape-memory alloy, preferably nitinol, arranged around the guidewire and temporarily fixed to the guidewire 42.

Once the distal portion 40 has reached its stimulation position (upper drawing of FIG. 6), withdrawal of the guidewire 42 toward the outside of the body causes the distal portion of the tube 43 to adopt the contact shape 430 (lower drawing of FIG. 6).

In the example shown, the contact shape 430 is a helix shape that extends inside the renal artery (RA) along the sympathetic nervous system S.

The diameter of the guidewire 42 can be, for example, 0.3556 mm (0.014 inch) or 0.4572 mm (0.018 inch). This diameter corresponds to the usual diameter that is already marketed and that allows stents, balloons or other stimulation or denervation devices to be conveyed within the renal or carotid arteries, whether they use a radiofrequency technique, ultrasound or injection of alcohol.

According to an advantageous embodiment, the electrical stimulation element 4 can comprise stimulation markers positioned at different positions of the proximal portion 40.

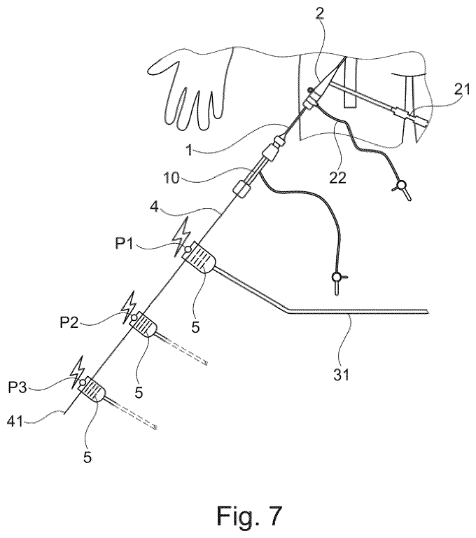

Such an embodiment is illustrated in FIG. 7: three markers implanted on the proximal portion 40 at three positions P1, P2, P3 spaced apart with a regular spacing, on each of which the stimulation clip 5 can be positioned. Thus, knowing the precise distance between the contact shape or shapes 400 to 405 and one of the positions P1 to P3, it is possible to know precisely the region of the nervous system S that will be stimulated.

It is thus possible to carry out true mapping of the regions of the nervous system S which may or may not be sensitive to the electrical stimulation performed with the contact shape 400 to 405.

This can make it possible to know precisely the location of the region or regions that are to be denervated subsequently.

And then, once the denervation procedure has been carried out, to accurately observe the region or regions that are effectively denervated.

We now describe the method of renal nerve stimulation and, where appropriate, of denervation, implemented by the kit described above.

This method is applied when a practitioner wishes to make a diagnosis of the renal artery, notably with a view to denervation.
Step i/: a nurse or a practitioner proceeds to fit the electrically conductive sleeve 2 around a guide catheter 1.
Step ii/: the practitioner then introduces the guide catheter 1 into a femoral artery of a human body C, so that the conductive periphery of the sleeve 2 is in contact with the subcutaneous tissue of the body or with the wall of the artery or vein.
Step iii/: the practitioner then introduces the wire 4 into the introducer sheath 11 of the guide catheter 1, until it is ensured that the distal portion 40 with its contact shape 400 is correctly positioned in the renal artery AR that is to be diagnosed.

The practitioner can then proceed with the electrical connection to the external electrical generator 3.

Thus, the nurse or the practitioner connects, on the one hand, the sleeve 2 to an electrode, typically the anode of the external cardiac stimulator 3, and, on the other hand, the proximal portion 41 of the wire 4 to the other electrode of the external generator 3.
Step iv/: Direct unipolar stimulation is performed on the wire 4. More precisely, the wire 4 conducts current in unipolar mode. The current delivered can be of the order of 15 mA to 25 mA with a frequency of between 200 and 800/min (10 to 20 Hz) for 1 min.

The response to this unipolar stimulation is then measured for each region of the nervous system S facing the region in contact with the contact shape 400 to 405 of the wire 4.

The renal nerve stimulation kit, i.e. for stimulation of the sympathetic nervous system around a renal artery, which has just been described can also be used to perform renal denervation.

Indeed, at the end of step iv/, if a nerve region S has been identified as responding notably to the stimulation, then, by choosing a guidewire, for example the guidewire 42 which also incorporates a device for emitting radiofrequency signals, ultrasound or for alcohol injection, it is possible to perform the renal denervation according to a step v/without removing any of the components of the kit.

The kit can also constitute a denervation control kit, because once the latter has been carried out, it is possible to again carry out unipolar electrical stimulation of each region assumed to be denervated by means of a contact shape 400 to 405, in order to verify that the denervation action has indeed been carried out.

With the same components described and a guide catheter 1 and an electrical stimulation element 4 adapted to the carotid anatomy, it is possible to produce a carotid nerve stimulation kit as shown schematically in FIG. 8.

Steps i/to iv/remain the same, with a contact shape 400 to 405 in contact with the carotid body.

In this use, the stimulation kit according to the invention can also constitute a kit for delivery of a carotid dilation stent or balloon. Step v/therefore consists in delivering a carotid dilation stent or balloon.

The invention is not limited to the examples just described; in particular, features of the examples illustrated may be combined with one another in alternatives not shown.

Other alternatives and improvements may be envisioned without thereby departing from the scope of the invention.

Even though, in the examples illustrated, the introduction of the guide catheter or introducer is done by the femoral route, that is to say into the femoral artery at the groin, it can also be done by the radial route, that is to say into the radial artery at the patient's wrist.

Instead of a guide catheter, an introducer can be used.

### LIST OF CITED REFERENCES

[1]: Pierre C. Qian et al. “Transvascular Pacing of Aorticorenal Ganglia Provides a stable Procedural Endpoint for Renal Artery Denervation”, Cardiovascular Interventions, vol. 12, no. 12, 2019, by the American College of Cardiology Foundation.
[2]: Masaomi Chinushi, et al. “Blood Pressure and Autonomic Responses to Electrical Stimulation of the Renal Arterial Nerves Before and After Ablation of the Renal Artery” Hypertension. 2013; 61:450-456. http://hyper.ahajournals.org
[3]: Konstantinos P. Tsioufis et al. “Safety and performance of diagnostic electrical mapping of renal nerves in hypertensive patients” EuroIntervention 2018; 14: E1334 e1342 published online September 2018. DOI: 10.4244/EIJ-D-18-00536

1. A kit for transvascular stimulation of part of an autonomic nervous system of a human body, comprising:
an introducer or a guide catheter, intended to be introduced into a blood vessel of the human body;
a sleeve adapted to be fitted around the introducer or guide catheter, the sleeve being made of electrically conductive material over at least part of its outer periphery so that, when the guide catheter is introduced into the blood vessel, the conductive periphery of the sleeve is in contact with the subcutaneous tissue of the human body or with the wall of the blood vessel, the sleeve additionally being connected to an electrical connection which is itself connected to an electrode of an electrical pulse generator external to the body;
an elongate electrical stimulation element intended to be inserted into the introducer or into the introducer sheath of the guide catheter, the elongate element comprising:
an electrically conductive distal portion, atraumatic to the blood vessel, and configured to be shaped with at least one point of contact with the blood vessel when it is in its stimulating position facing the part of the autonomic nervous system around the blood vessel,
an electrically insulating central portion, and
an electrically conductive proximal portion serving for connection to the other electrode of the electrical pulse generator, so as to effect the electrical stimulation through the shaped distal portion.

2. The stimulation kit as claimed in claim 1, the electrode of the electrical pulse generator to be connected to the electrically conductive sleeve fitted around the introducer or the introducer sheath of the guide catheter being the anode, while the one to be connected to the proximal portion of the elongate element is the cathode.

3. The stimulation kit as claimed in claim 1, in which the electrically conductive sleeve is composed of a one-piece component made of conductive material.

4. The stimulation kit as claimed in claim 3, the sleeve being composed of a sheath having an electrically conductive coating, on its outer periphery.

5. The stimulation kit as claimed in claim 3, the sleeve being elastic so as to be able to fit on peripheral arterial or venous catheter sheaths of different diameters, typically of external diameters of between 1.6 and 20 mm.

6. A kit for transvascular stimulation of part of an autonomic nervous system of a human body, comprising:
an introducer or a guide catheter intended to be introduced into a blood vessel of the human body, the introducer or the guide catheter comprising at least one tubular introducer sheath and at least one electrically conductive element, of which a distal portion is exposed over at least part of the outer periphery of the sheath so as to be in contact with subcutaneous tissue of the body or with the wall of the blood vessel, and of which a proximal portion, accessible from outside of the body, comprises an electrical connection so as to serve as a connection to an electrode of an electrical pulse generator to the body;
an elongate electrical stimulation element intended to be inserted into the introducer or into the introducer sheath of the guide catheter, the elongate element comprising:
an electrically conductive distal portion, atraumatic to the blood vessel, and configured to be shaped into a shape with at least one point of contact with the blood vessel when it is in its stimulating position facing the part of the autonomic nervous system around the blood vessel,
an electrically insulating central portion, and
an electrically conductive proximal portion for connection to the other electrode of the electrical pulse generator, so as to effect the electrical stimulation through the shaped distal portion.

7. The stimulation kit as claimed in claim 6, the electrode of the electrical pulse generator to be connected to the introducer or to the guide catheter being the anode, while the one to be connected to the proximal portion of the elongate element is the cathode.

8. The stimulation kit as claimed in claim 6, in which the electrically conductive element of the introducer or of the guide catheter is a wire or a metal strip which is housed at least partly within the thickness of the sheath and of which a distal portion is exposed at the outer periphery of the sheath.

9. The stimulation kit as claimed in claim 8, the cross section of the wire or of the metal strip being between 0.25 and 5 mm².

10. The stimulation kit as claimed in claim 1, the elongate element being a rectilinear wire, the distal portion of which comprises the contact shape, which extends radially along the axis of the wire.

11. The stimulation kit as claimed in claim 10, the contact shape comprising at least one loop at the end of the wire or in proximity to the straight end of the wire.

12. The stimulation kit as claimed in claim 10, the contact shape comprising at least one ring in proximity to the straight end of the wire.

13. The stimulation kit as claimed in claim 10, the contact shape comprising at least one free strand in proximity to the straight end of the wire.

14. The stimulation kit as claimed in claim 1, the elongate element comprising a guidewire, and a tube made of shape-memory alloy arranged around the guidewire and temporarily attached to the guidewire so that, once its distal portion has reached its stimulation position, withdrawal of the guidewire toward the outside of the body causes the distal portion of the tube to adopt the contact shape.

15. The stimulation kit as claimed in claim 14, the contact shape of the distal portion of the tube being a helix shape.

16. The stimulation kit as claimed in claim 1, the distal portion of the elongate element comprising one or more radiopaque markers.

17. The stimulation kit as claimed in claim 1 being a kit for transvascular renal nerve stimulation, and furthermore a renal denervation kit.

18. The stimulation kit as claimed in claim 1, being a kit for stimulation of the carotid body, and furthermore a kit for delivery of a stent or a balloon for carotid dilation.
